(19) United States
(12) Patent Application Publication
(10) Pub. No.: US 2016/0166627 A1
(43) Pub. Date: Jun. 16, 2016

(54) JOINT HEALTH COMPOSITIONS

(71) Applicants: Liya Yan DENNEY, Lausanne 26 (CH); Haotian FENG, Beijing (CN); Marie Noelle HORCAJADA, Lausanne 26 (CH); Elizabeth OFFORD CAVIN, Lausanne 26 (CH); NESTEC S.A., Vevey (CH)

(72) Inventors: Liya Yan Denney, Lausanne 26 (CH); Haotian Feng, Beijing (CN); Marie Noelle Horcajada, Lausanne 26 (CH); Elizabeth Offord Cavin, Lausanne 26 (CH)

(21) Appl. No.: 14/902,763

(22) PCT Filed: Jul. 5, 2013
(86) PCT No.: PCT/CN2013/078890

§ 371 Date: Jan. 4, 2016

(51) Int. Cl. A61K 36/40; A61K 36/488

(52) U.S. Cl. CPC A61K 36/40; A61K 36/488; A23L 1/3002; A23V 2002/00

(57) ABSTRACT

Joint health compositions for maintaining mobility and cartilage and preventing osteoarthritis, one of the compositions comprises Fructus Corni, said composition further comprises a Kudzu extract and/or a source of protein, a source of calcium and a source of vitamin D, another composition comprises Fructus Corni aqueous extract and milk powder.

The present invention relates generally to compositions with a health benefit. In particular the invention relates to the field of joint health. A subject matter of the invention is a composition comprising fructus corni for use in the maintenance of joint health in mammals. A further aspect of the invention is a composition comprising fructus corni aqueous extract and milk powder.

Joint disorders are the most frequent cause of disability among older adults. The loss of joint health makes it very difficult for individuals to be physically active, contributing to an increased risk of obesity and reduced quality of life. Older people with joint disorders often become house-bound. Many joint disorders involve joint inflammation (arthritis). Osteoarthritis (sometimes called degenerative arthritis) is the most common joint disorder, often showing symptoms in people in their 40s and 50s and becoming nearly universal (although not always symptomatic) by age 80. Osteoarthritis can affect both the larger and the smaller joints of the body, including the hands, feet, back, hip, and knee. Osteoarthritis is essentially acquired from daily wear and tear of the joints; however, osteoarthritis can also occur as a result of injuries such as sports injuries. Osteoarthritis is characterized by disruption and loss of joint cartilage along with other joint changes.

Joint disorders affect animals as well as humans. From a pet perspective, osteoarthritis is very common in older cats [D. Bennett et al., J Feline Med. Surg., 14, 65-75 (2012)] and it has been estimated that one in five adult dogs is affected by osteoarthritis [Veterinary Technician, 24, 664-675 (2003)]. Osteoarthritis causes limitation of movement as well as tenderness, inflammation, and pain—all of which can greatly affect a pet's quality of life and cause anxiety and distress to the pet's owner.

There is currently no pharmacological cure for osteoarthritis and it is very difficult to restore the cartilage once it is destroyed. Most therapy focuses on pain relief using non-steroidal anti-inflammatory drugs (NSAIDs). It would therefore be beneficial to find an effective composition for the maintenance of joint health, for example a composition which can prevent osteoarthritis in healthy individuals, or which can slow its development.

Glucosamine has attained great popularity as a nutritional supplement, primarily for joint health. Animal models and in vitro experiments suggest that glucosamine may both stimulate cartilage production and inhibit cartilage degradation. Glucosamine sulphate has been reported to be able to significantly reduce the symptoms of osteoarthritis in the lower limbs [Rheumatology International, 32, 2959-2967 (2012)] and EP1890709 describes how glucosamine can be generated from plants such as chicory, carrot, and Jerusalem artichoke. However, opinion on the effectiveness of glucosamine is divided.

Fructus corni is the fruit of Cornus officinalis, a species of dogwood also known as Japanese cornelian cherry, which grows mainly in China, Japan and Korea. Fructus corni has been widely used as a traditional Chinese medicine for over 2000 years, with reported effects including lowering blood lipids, controlling postprandial hyperglycemia and improving male sexual function. Fructus corni is typically used in herbal medicine as the dried sarcocarp of the fruit. The fruit is dipped in boiling water for several minutes, then the core is removed, and the seedless fruit is dried to obtain fructis corni. Fructus corni contains glycosides such as morroniside and loganin. Fructus corni is produced commercially and is permitted for use as a functional food in China.

WO2005/003145 describes using an extract of Cornus officinalis for treating pain, inflammation and autoimmune disorders. One of the autoimmune disorders listed is rheumatoid arthritis, the extract of Cornus officinalis providing an immunosuppressive effect. Guo et al. describe the effects of an extract of Fructus corni on an in-vitro model for diseased joints [Guo Lili Zhou Yong and Wang Xudan, Effects of total glycoside from Shanzhuyu (the sarocarp of Cornus officinalis) on chemotaxis of the supernatant of cultured primary synoviocytes of arthritic rats, Journal of Beijing University of Traditional Chinese Medicine, 2002(03):30˜2]. There have been no reports that an extract of Cornus officinalis might maintain joint health in healthy subjects.

There is a need to find new compositions for use in the maintenance of joint health; in particular compositions which reduce or prevent cartilage degeneration in healthy individuals. It would be especially valuable to identify further compositions from natural sources, and compositions which are suitable for inclusion in a regular diet.

The object of the present invention is to improve the state of the art and to provide an improved solution for the maintenance of joint health, or at least to provide a useful alternative. The object of the present invention is achieved by the subject matter of the independent claims. The dependent claims further develop the idea of the present invention. Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”.

The present invention provides in a first aspect a composition comprising fructus corni for use in the maintenance of joint health in mammals. In a second aspect, the invention relates to a composition comprising between 1 and 30% fructus corni aqueous extract and between 40 and 99% of milk powder. Unless otherwise stated, all percentages in the current specification are on a weight basis.

Mature ovariectomized rats provide a model for the deterioration of joints which occurs in healthy mammals [Høegh-Andersen et al., Arthritis research & therapy 6 (2), R169-180 (2004)]. The inventors surprisingly found that fructus corni had positive effects against cartilage degeneration in mature ovariectomized rats. Fructus corni water extract was found to decrease the concentration of CTX-II (cross linked C-telopeptide of type 2 collagen), a biomarker of cartilage degeneration. In addition, treatment with fructus corni water extract was found to decrease the inflammation marker TNF-α (tumor necrosis factor α) in serum from the ovariectomized rats. These findings indicate that fructus corni can provide protective effects on joint degeneration in healthy individuals, as well as providing an anti-inflammatory effect. A composition comprising fructis corni may therefore be effectively used in the maintenance of joint health in mammals, for example healthy mammals.

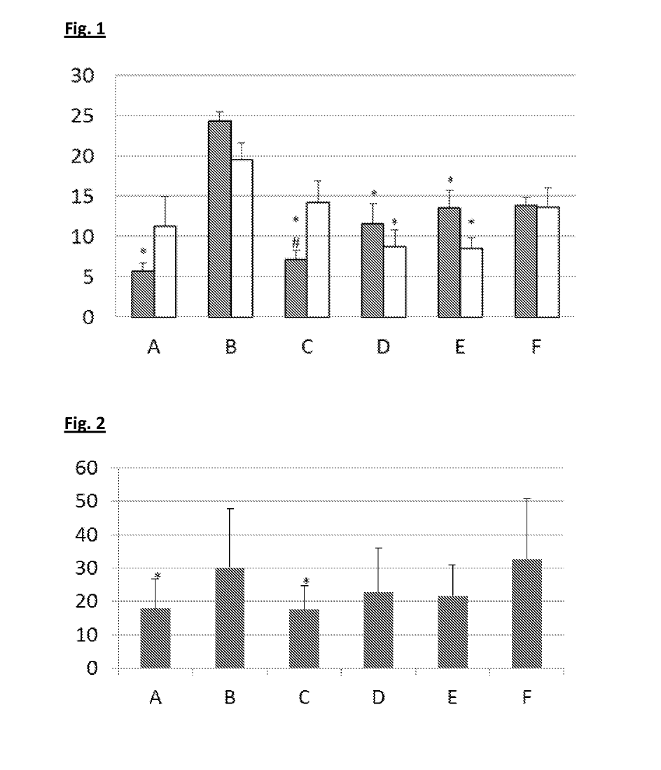

FIG. 1 shows serum CTX-II concentration (cartilage degeneration marker) in pg/ml at week 6 ▪ and at week 12 □ for; sham surgery (A), ovariectomized (B), ovariectomized with fructus corni aqueous extract treatment (C), ovariectomized with fructus corni resin ethanol/water extract treatment (D), ovariectomized with fructus corni aqueous water extract and kudzu water extract treatment (E) and ovariectomized with glucosamine treatment (F). * indicates P<0.01 vs. ovariectomized. # indicates P<0.01 vs. glucosamine.

FIG. 2 shows serum TNF-α in pg/ml for; sham surgery (A), ovariectomized (B), ovariectomized with fructus corni aqueous extract treatment (C), ovariectomized with fructus corni resin ethanol/water extract treatment (D), ovariectomized with fructus corni aqueous water extract and kudzu water extract treatment (E) and ovariectomized with glucosamine treatment (F). * indicates P<0.05 vs. ovariectomized.

The invention provides a composition comprising fructis corni for use in the maintenance of joint health in mammals. Healthy joints are very important for having an active and pain free life. Without healthy joints, problems of mobility can lead to other detrimental conditions such as depression and obesity. Maintenance of joint health may be for example the prevention or reduction of joint degeneration which occurs over time due to everyday wear and tear.

Osteoarthritis, also known as degenerative arthritis or degenerative joint disease is a group of mechanical abnormalities involving degradation of joints. Osteoarthritis can affect any joint, but hands and weight-bearing joints—including the spine, hips and knees—are most often affected. Other joints, like shoulders, elbows, and ankles, are also likely to be affected. In humans it strikes most commonly after the age of 45, but people of all ages—from children to senior citizens-are at risk. It is likely that most mammals who survive to old age will suffer from osteoarthritis. For example, osteoarthritis is known in horses, dogs, cats, and primates. The composition of the invention may be for use in the maintenance of joint health in mammals where joint health is maintained by the prevention of osteoarthritis.

A composition comprising fructis corni may be for use in maintaining the mobility of a mammal. The composition helps to maintain joint health, which results in a better activity or mobility of the individual. Administering the composition comprising fructis corni to a human, a pet or a horse greatly improves their quality of life. In the case of pets, better mobility enriches and improves the interaction between the pet and its owner.

Osteoarthritis is commonly caused by the breakdown of cartilage in the joints. Fructis corni reduces cartilage degeneration in humans or other mammals. Cartilage is a flexible connective tissue found in many areas in the bodies of humans and other animals, including the joints between bones, the rib cage, and the intervertebral discs. Cartilage degeneration is a critical aspect of loss of joint health. For example, cartilage degeneration occurs in osteoarthritis. A composition comprising fructis corni may be for use in maintaining cartilage.

Fructis corni may be provided in any form suitable for administration to humans or animals. For example, fructis corni may be provided as a dry powder or as an extract.

The composition of the invention may be provided as an aqueous extract. Fructis corni aqueous extract may be obtained by extracting dried fructis corni with water, for example boiling water; after filtration the extract is concentrated and dried to a powder.

The composition of the invention may further comprise other materials associated with maintaining joint health such as glucosamine and/or chondroitin sulphate. The composition of the invention may further comprise Kudzu extract. Kudzu (Pueraria lobata) is a climbing vine which originated in China and was brought to the United States from Japan in the late 1800s. It is distributed throughout much of eastern United States and is most common in the southern part of the continent. Kudzu may be provided in any form suitable for administration to humans or animals. Kudzu root is known to reduce articular cartilage degradation [WO2011/135011] and so may complement fructis corni when taken together, for example allowing lower dosages of each to achieve the same effect.

The composition of the invention may be any composition that is suitable for human or animal consumption. As such the composition may be selected from the group consisting of a food, a drink, a nutritional supplement, a nutritional formula, and a pet food product. A nutritional supplement, also known as food supplement or dietary supplement, is a preparation intended to supplement the diet and provide nutrients, such as vitamins, minerals, fibre, fatty acids, or amino acids that may be missing or may not be consumed in sufficient quantities in a person's diet. Nutritional supplements are commonly taken orally in the form of a tablet.

The nutritional formula may be a complete nutritional formula which provides sufficient types and levels of macronutrients (protein, fats and carbohydrates) and micronutrients to be sufficient as a sole source of nutrition for the subject to which it is administered. The nutritional formula may also provide partial nutrition, to act as a supplement to the existing diet of the subject.

The composition of the invention may be administered orally, enterally or topically. Most people and animals dislike having injections and so it is an advantage to have a composition which can be administered without an injection.

The composition of the invention may be administered to an athlete. Athletes who train, practice, and play sports that range over the novice level, are more likely to develop poor joint health, for example osteoarthritis, than those individuals that exercise at a more moderate level. Participation in vigorous sports, such as football, rugby, and track and field events can result in joint stress and injuries. These may lead to cartilage degeneration, possibly in later life.

The composition of the present invention may be to be administered to adults or the elderly. This is advantageous as joint health often diminishes with age. For the purpose of the present invention a subject shall be considered as “elderly” if it has surpassed the first half of its average expected lifespan in its country of origin, preferably, if it has surpassed the first two thirds of the average expected lifespan in its country of origin, more preferably if it has surpassed the first three quarters of the average expected lifespan in its country of origin, most preferred if it has surpassed the first four fifths of the average expected lifespan in its country of origin. A composition for use according to the invention may be administered to adult humans over 40 years old, for example over 50 years old, for further example over 60 years old.

As deteriorating joint health not only occurs in humans but also in other animals, the composition of the present invention may be to be administered to humans or animals, for example companion animals such as cats or dogs.

The compositions of the present invention are effective following a dose-response curve. They may be administered in an amount that is sufficient to at least partially reduce the risk of developing poor joint health. The precise amounts depend on a number of subject specific factors such as the subject's state of health and weight.

Skilled artisans will be able to determine such dosages appropriately. However, generally the composition of the present invention may be to be administered in a daily dose corresponding to at least 10 mg fructus corni on a dry basis per kg body weight per day. For example at least 20 mg fructus corni on a dry basis per kg body weight per day, for further example at least 50 mg fructus corni on a dry basis per kg body weight per day. To avoid unwanted side effects the dosages should be kept at a reasonably low level. The composition of the present invention may be administered in a daily dose corresponding to between 10 mg and 500 mg of fructis corni on a dry basis per kg body weight per day.

Different extraction methods from fructus corni may provide different concentrations of active ingredients, but the dose can be scaled according to the morroniside content. Morroniside content can be measured by the methods known in the art, for example Zhou et al. [Pharmaceutical Care and Research 12 (6), pp. 441+446+457 (2012)]. Dry fructis corni typically contains around 10 mg/g of morroniside. Accordingly, the composition of the present invention may be administered in a daily dose comprising at least 0.1 mg, 0.2 mg, or 0.5 mg morroniside per kg body weight per day. The composition of the present invention may be administered in a daily dose comprising between 0.1 mg and 5 mg of morroniside per kg body weight per day.

The composition comprising fructus corni for use in accordance with the invention may further comprise a source of protein, a source of calcium and a source of vitamin D. The source of protein may be selected from the group consisting of milk powder, whey protein, soy protein or mixtures of these. Protein aids in building muscles and calcium and vitamin D help build and maintain strong and healthy bones. Having healthy muscles, bones and joints leads to good mobility; and as a virtuous circle, the maintenance of good joint health is more effective in individuals with good mobility. A composition comprising fructus corni, protein, calcium and vitamin D is therefore beneficial in the maintenance of joint health in mammals.

The invention further provides a composition comprising between 1 and 30% fructus corni aqueous extract and between 40 and 99% of milk powder, for example between 5 and 25% fructus corni aqueous extract and between 50 and 95% of milk powder, for further example between 10 and 20% fructus corni aqueous extract and between 60 and 90% of milk powder. Fructus corni aqueous extract is generally formulated as a powder, for example spray-dried with a carrier such as maltodextrin. However, as the quantity of the carrier may vary, the level of fructus corni aqueous extract in the composition of the invention is given on a dry basis and does not include any carriers with which it may have been formulated. 100 g of the composition may comprise between 10 mg and 300 mg of morroniside. The milk may be skimmed, partially skimmed or whole milk. The composition comprising fructus corni aqueous extract and milk powder may further comprise vitamins and minerals. For example, 100 g of the composition may comprise between 2 and 25 μg of vitamin D; and between 1 and 3 g of calcium. The vitamin D may be cholecalciferol. Calcium and vitamin D help promote and maintain strong bones which is important for joint health. Good bone health promotes good joint health. For example, aging individuals with healthy bones are able to remain more active which benefits joint health. Combining vitamin D, calcium and fructus corni with milk provides a composition beneficial for joint health.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification. Further advantages and features of the present invention are apparent from the figures and non-limiting examples.

### EXAMPLE 1

### Preparation of Fructus corni Aqueous Extract (FCw)

12 kg of dried fructus corni (Bozhou Yonggang) was mixed with 144 L hot water in a 200 L extraction tank. The mixture was stirred at a temperature of 95° C. for 1 hour and then filtered through a 100 mesh stainless sieve. The residue of this extraction was then extracted with a further 60 L of hot water at 95° C. for 30 minutes before being filtered through a 100 mesh stainless sieve. The two extractions were combined, cooled to 35° C., and then centrifuged. The clear liquid obtained was concentrated by using a reverse osmosis membrane to obtain a concentrate with a Brix of approximately 5.5° Bx. This concentrate was then further condensed in a vacuum at 80° C. for 1 hour. The resulting condensed concentrate was blended with 6.5 kg dextrin and spray-dried. 12 kg of spray dried powder extract was manufactured, having a morroniside content of 10.8 mg/g.

### EXAMPLE 2

### Preparation of Fructus corni Resin Ethanol/Water Extract (FCr)

120 L of Amberlite™ XAD1600 resin (Rohm and Haas) was pre-treated by soaking in 1% NaOH for 12 hours, then washed to pH 6.5-7.5 using pure water, soaked in 95% ethanol/water for 12 hours and repeatedly washed with pure water to remove ethanol. 50 kg of dried fructus corni (Bozhou Yonggang) was mixed with 600 L hot water in a 3000 L extraction tank. The mixture was stirred at a temperature of 95° C. for 1 hour and then filtered through a 100 mesh stainless sieve. The residue of this extraction was then extracted with a further 250 L of hot water at 95° C. for 30 minutes before being filtered through a 100 mesh stainless sieve. The two extractions were combined, cooled to 35° C., and then centrifuged. 800 L of the clear liquid was loaded on 120 L of XAD1600 resin column with a flow rate of 240 L/h and then flushed with 300 L of pure water. The resin column was eluted by using 300 L of 70% ethanol/water, with a flow rate of 120 L/h. The ethanol/water fraction was collected and condensed under vacuum at 70° C. for 120 minutes to recycle the ethanol. This produced 50.4 kg of 6.8° Bx concentrate which was spray-dried (with no carrier) resulting in 3.15 kg of spray dried powder extract, having a morroniside content of 180 mg/g.

### EXAMPLE 3

### In vivo Study

Ovariectomized (OVX) rats were used as a model to mimic osteoarthritis in healthy postmenopausal women. The effect of fructus corni on suppressing markers of cartilage degeneration, cross linked C-telopeptide of type 2 collagen (CTX-II) and inflammation, tumor necrosis factor α (TNF-α), was examined.

Seven month-old female Sprague-Dawley rats were purchased from Beijing Weitong Lihua Laboratory Animal Centre. Surgery was performed by the research team in Shenyang Medical College. The rats were anaesthetized with Hypnorm™/Dormicum™ (1 part Hypnorm™+1 part Dormicum™+2 parts sterile water) given by injection at 0.2 ml/100 g body weight. After anaesthesia the animal was shaved on the back and the skin disinfected. A single incision in the skin was made above the spine, providing access to both ovaries. The muscles were cut to provide access and the ovaries were located and removed by cutting. The muscle on each side was closed with 1-2 stitches, and the skin was closed with 3-4 stitches. For sham-operation, the same procedure for OVX-surgery was followed with the exception that the ovaries were not cut and removed. The animals were given post-operational analgesia (Rimadyl™) on the day of operation and the following two days.

The animals were kept in temperature and humidity controlled rooms (22±2° C., 50±20% humidity) with a 12 h/12 h light/dark cycle. The animals had a 1 week acclimatization period between the operation and the start of the experimental treatment. The treatment lasted for a 12 week period. The animals were body-weight-matched and were randomly assigned to the following groups:

| Treat- | | | | |
|---|---|---|---|---|
| ment | | | Treatment | No. of |
| Group | Surgery | Diet | dose | Animals |
| A | Sham | AIN-93M diet | — | 10 |
| B | OVX | AIN-93M diet | — | 10 |
| C | OVX | AIN-93M diet + 2000 mg/kg | 5% FCw | 10 |
| | | Fructus corni aqueous extract | in diet | |
| D | OVX | AIN-93M diet + 200 mg/kg | 0.5% FCr | 10 |
| | | Fructus corni resin ethanol/ | in diet | |
| | | water extract | | |
| E | OVX | AIN-93M diet + 2000 mg/kg | 5% FCw + | 10 |
| | | Fructus corni aqueous | 0.75% Ku | |
| | | extract + 300 mg/kg Kudzu | in diet | |
| | | (Ku) water extract | | |
| F | OVX | AIN-93M diet + 300 mg/kg | 0.5% GS in | 10 |
| | | glucosamine (GS) | drinking | |
| | | | water | |

Food was AIN-93M diet [P. G. Reeves et al., Journal of Nutrition 123 (11), 1939 (1993)]. Plant extracts was administered via food. Kudzu water extract was obtained from High Star Ltd. The extract for each test group was homogenously mixed with food according to the dose and estimated average food intake. Food pellets were made after mixing. The amount of food given to the rats was 90% of their mean food intake to insure the rats eat all the food fed. Glucosamine (GS) hydrochloride (Regenasure®glucosamine, Cargill, US) was used as a positive control for anti-cartilage degeneration and administrated via deionized drinking water. The rats were kept two to a cage. The amount of diet was 16 g/day per rat of the respective diet, the minimal average food intake of the rats during the acclimation period. All rats had free access to distilled water. Rats in group F had free access to distilled water containing 0.5% glucosamine and the amount of water consumed was recorded for each rat. The treatment with test diets started one week after ovariectomy and lasted for 12 weeks. Supernumerary animals were operated for each group in case of replacement of individuals during the first week of treatment.

1.5 ml blood samples were collected at 0, 6 and 12 weeks from all animals. Serum was then prepared by centrifugation of the collected blood (3000 rpm for 10 min). The serum fraction was carefully collected into labelled Eppendorf™-tubes and kept frozen at −80° C. for later analysis. At the end of the study the rats were anaesthetised with isoflurane inhalation before being euthanized by CO₂ inhalation.

CTX-II was measured using the Serum Preclinical Cartilaps ELISA (Nordic Bioscience, Herlev, Denmark). The intra- and inter-assay coefficient variations (CVs) were 4.6% and 16.2% respectively. Serum TNFα was measured using Rat TNF-α Platinum ELISA (eBioscience, USA) for the serum collected at the end of the study only. Intra- and inter-assay CVs were 3.5% and 9.7% respectively.

Data were expressed as means±SD. All statistical analyses were performed with SPSS 13.0 statistic software (SPSS Inc, Chicago, Ill., USA). One-way ANOVA was used to test for differences among groups.

At week 0, the CTX-II concentration in serum was not significantly different between groups. At week 6, serum CTX-II concentration was significantly increased in OVX group (B) compared to Sham group (A) (P<0.01), demonstrating the success of this model (FIG. 1). The OVX induced increase in CTX-II was significantly suppressed in all treatment groups (P<0.01) at week 6. At week 12, the difference in CTX-II between the sham and OVX was smaller but the suppression in CTX-II could still be observed in groups D (Fructus corni resin ethanol/water extract) and E (Fructus corni aqueous extract+kudzu).

At week 12, serum TNF-α concentration was significantly increased in OVX group (B) compared to Sham group (A) (P<0.05) (FIG. 2). Fructus corni aqueous extract treatment (C) suppressed the increase in TNF-a induced by OVX (P<0.05) but this was not observed in other treatment groups including the positive control glucosamine group (F).

Fructus corni treatment decreased the marker of cartilage degeneration CTX-II in mature OVX rats. This effect was observed for the aqueous extract, the resin ethanol/water extract and the aqueous extract combined with kudzu water extract. The magnitude of effect was as good as its positive control, glucosamine. In addition, Fructus corni aqueous extract also decreased the serum inflammation marker TNF-α in serum in the OVX rats whereas this effect was not observed with glucosamine. Reducing inflammation is an additional benefit, as inflammation generally occurs together with cartilage damage and worsens the subject's overall joint health. These findings show that fructus corni can provide protective effects on joint degeneration, for example in aging. This demonstrates that a composition comprising fructus corni may be for use in the maintenance of joint health in mammals.

1. A method for the maintenance of joint health in mammals comprising administering a composition comprising fructus corni to a mammal in need of same.

2. Method in accordance with claim 1 where joint health is maintained by the prevention of osteoarthritis.

3. Method in accordance with claim 1 for use in maintaining the mobility of a mammal.

4. Method in accordance with claim 1 for use in maintaining cartilage.

5. Method in accordance with claim 1 wherein fructus corni is provided as an aqueous extract.

6. Method in accordance with claim 1 wherein the composition further comprises Kudzu extract.

7. Method in accordance with claim 1 wherein the composition is in a form selected from the group consisting of a food, a drink, a nutritional supplement, a nutritional formula, and a pet food product.

8. Method in accordance with claim 1 wherein the mammal is a human.

9. Method in accordance with claim 1 wherein the composition is to be administered to adults or the elderly.

10. Method in accordance with claim 1 wherein the mammal is an adult human over 40 years old.

11. Method in accordance with claim 1 wherein the mammal is an athlete.

12. Method in accordance with claim 1 wherein the composition is to be administered in a daily dose corresponding to at least 10 mg fructus corni on a dry basis per kg body weight per day.

13. Method in accordance with claim 1 wherein the composition further comprises a source of protein, a source of calcium and a source of vitamin D.

14. Composition comprising between 1 and 30% fructus corni aqueous extract and between 40 and 99% of milk powder

15. Composition according to claim 14 wherein 100 g of the composition comprises between 2 and 25 μg of vitamin D and between 1 and 3 g of calcium.
